(19) United States
(12) Patent Application Publication
(10) Pub. No.: US 2022/0192491 A1
(43) Pub. Date: Jun. 23, 2022

(54) OPHTHALMIC CART AND METHOD

(71) Applicant: HealTheia, LLC, Winter Park, FL (US)

(72) Inventors: Samuel K. Steven Houston, III, Winter Park, FL (US); Ehsan Rahimy, San Francisco, CA (US); John Brown Miller, Winchester, MA (US)

(21) Appl. No.: 17/556,205

(22) Filed: Dec. 20, 2021

Related U.S. Application Data
(60) Provisional application No. 63/128,546, filed on Dec. 21, 2020.

(51) Int. Cl. A61B 3/18; A61B 3/16; A61B 3/12; A61B 3/14; A61B 3/00; A61B 3/10

(52) U.S. Cl. CPC A61B 3/18; A61B 3/16; A61B 3/102; A61B 3/14; A61B 3/005; A61B 3/12

(57) ABSTRACT

An ophthalmic and associated methods are disclosed. In one example a plurality of ophthalmic tools are located on a single cart for collecting ophthalmic data. Data may be recorded in a standardized format to aid in comparison between records in a database. In one example the standardized format facilitates fast and more accurate diagnoses of ophthalmic conditions.

### PRIORITY

This application claims the benefit of priority to U.S. Provisional Patent Application Ser. No. 63/128,546 filed Dec. 21, 2020, which is incorporated by reference herein in its entirety.

### TECHNICAL FIELD

Embodiments described herein generally relate to devices and methods for ophthalmic data collection, diagnosis, and analysis.

### BACKGROUND

When a patient visits a first level physician, such as a primary care doctor, with symptoms pertaining to an eye condition, the first level physician often requests the expertise of a specialist for timely and appropriate evaluation. An ophthalmologist, or other eye specialist has the requisite skill necessary to diagnose ocular conditions, and to decide an appropriate course of action. However, the ophthalmic data required to make an accurate diagnosis frequently requires specific testing and examination equipment not available to the first level physician. The patient may be sent to see an ophthalmologist, or other eye specialist only to find out that once the appropriate data is obtained and tabulated by the specialist, that the condition does not require specific in-office treatment. In such a case, the travel to the specialist is wasted time for the patient and increased use and cost of healthcare resources. A similar scenario is often encountered in surgery, where a surgeon in a remote area may encounter a surgical case that additional expertise is warranted to ensure the best possible outcome for a patient, whether the surgeon and patient are in an underserved area of the United States, or in any area around the World. Accordingly, devices and methods for improved patient evaluation in a remote and virtual clinical and surgical setting are desired.

### BRIEF DESCRIPTION OF THE DRAWINGS

FIG. 1 shows an ophthalmic cart in accordance with some example embodiments.

FIG. 2 shows one component from an ophthalmic cart in accordance with some example embodiments.

FIG. 3 shows another component from an ophthalmic cart in accordance with some example embodiments.

FIG. 4A shows another component from an ophthalmic cart in accordance with some example embodiments.

FIG. 4B shows imaging capability of the component from FIG. 4A in accordance with some example embodiments.

FIG. 5 shows an eye chart for use with an ophthalmic cart in accordance with some example embodiments.

FIG. 6 shows a block diagram of software functionality for use with an ophthalmic cart in accordance with some example embodiments.

FIG. 7 shows a flow diagram of a method of collecting and utilizing ophthalmic data in accordance with some example embodiments.

FIG. 8 shows a block diagram illustrating an example machine upon which any one or more of the techniques (e.g., methodologies) discussed herein may be performed, in accordance with some example embodiments.

### DESCRIPTION OF EMBODIMENTS

The following description and the drawings sufficiently illustrate specific embodiments to enable those skilled in the art to practice them. Other embodiments may incorporate structural, logical, electrical, process, and other changes. Portions and features of some embodiments may be included in, or substituted for, those of other embodiments. Embodiments set forth in the claims encompass all available equivalents of those claims.

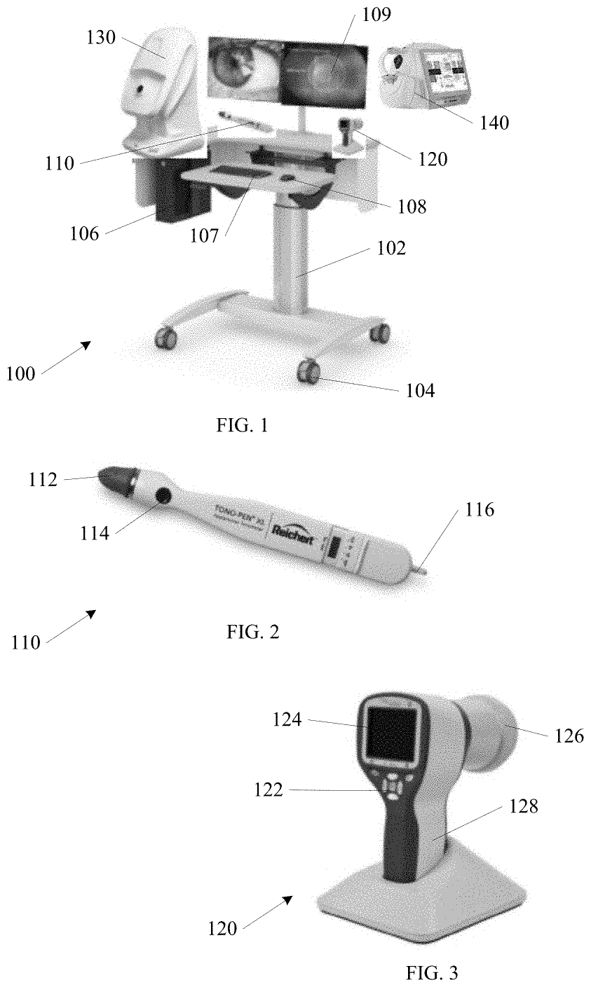

FIG. 1 shows one example of an ophthalmic cart 100. The cart 100 includes a base 102, and wheels 104, although the invention is not so limited. It may be less expensive for a clinic to have only one cart 100 as many of the ophthalmic tools are expensive. The addition of wheels 104 makes easy movement of the cart 100 between different examination rooms. In one example, the cart 100 further includes a computer 106 with a keyboard 107 and a mouse 108, and a display screen 109. In one example, the computer 106 and associated peripherals (107, 108, 109) are used to run software and collect ophthalmic data from a number of different ophthalmic diagnostic tools discussed in more detail below. In one example, software stored or otherwise executed by the computer 106 may be used to instruct a user about operation of the ophthalmic tools for a user that may be less familiar with their operation.

The ophthalmic cart 100 includes a plurality of ophthalmic tools on a single cart 100 for collecting ophthalmic data in a consistent and methodical manner, and in a designed sequence to enable remote evaluation and management, as well as the development of artificial intelligence and machine learning tools to provide complex decision-making on a breadth of eye conditions. In the example of FIG. 1, the ophthalmic cart 100 includes four ophthalmic tools although the invention is not so limited. Other numbers of ophthalmic tools fewer than three or more than three are within the scope of the invention.

The collection of ophthalmic tools on a single cart facilitates a more thorough examination by a first level physician, such as a primary care or emergency room physician, who may not be familiar with ophthalmic testing. In one example, the first level physician is enabled to gather enough data and imaging without any eye specific experience at a collection location, such as the first level physician's office, urgent care center, or emergency room. In one example, the collection of ophthalmic tools is specifically selected to provide enough ophthalmic data about a patient, in a specified and useful data format, to diagnose an ophthalmic condition from an outside, remote eye care specialist. In a similar manner, video data capture of surgical video and data will be transmitted from a remote location to the cart. An expert surgeon can then remotely proctor or train the remote surgeon live during a surgical procedure through remote viewing tools and real-time, audio-video communication.

In examples shown, the collected ophthalmic data is transmitted to a second location, remote from the collection location. For example, the collected ophthalmic data may be transmitted to a specialist's location, such as an ophthalmologist or a vitreoretinal surgeon, etc. In one example, ophthalmic tool parameters are selected to be consistent between a number of carts 100 at different locations. In one example ophthalmic tool types are similar or identical for each of several carts 100. In one example ophthalmic tool examination settings are similar or identical for each of several carts 100.

In one example, for any single given cart 100, ophthalmic tool parameters are selected to be consistent with a database standard. A single cart 100 may be used to evaluate a number of patients, or a number of carts 100 may each contribute records to the database. It is desirable to have consistent data from the ophthalmic tool types that may be used to compare a selected patient quantitatively to other patients in the database. Standardization of tool parameters is discussed in more detail below.

The ophthalmic cart 100 includes an intraocular pressure measurement device 110. In one example, the intraocular pressure measurement device 110 includes a Tono-Pen. Although a Tono-Pen is used as an example, other intraocular pressure measurement devices 110 are within the scope of the invention. Different intraocular pressure measurement devices 110 may be used from one cart 100 to the next, provided that consistent ophthalmic data is obtained between devices 100. For example, if a Tono-Pen provides the same accurate intraocular pressure as a non-Tono-Pen intraocular pressure measurement device 110 then different devices 110 are considered to provided standardized data.

The ophthalmic cart 100 further includes an external imaging device 120. In one example, the external imaging device 120 includes an anterior segment camera, such as a Volk Pictor or the like. In one example the external imaging device 120 provides high-resolution images of the surface of the eye and areas directly surrounding the eye. In one example, cobalt blue LED light allows fluorescent imaging to detect a dry eye or any trauma on the ocular surface. Although an anterior segment camera is used as an example, other external imaging device 120 are within the scope of the invention, including cameras in smartphones or tablets with or without macro lens add-ons.

Similar to the intraocular pressure measurement device 110 discussed above, different external imaging devices 120 may be used from one cart 100 to the next, provided that consistent ophthalmic data is obtained between devices 120. For example, if an anterior segment camera provides the same image resolution and/or color accuracy, etc. as other external imaging devices 120 then different devices 120 are considered to provided standardized data. In one example, image capture settings are specified for any given external imaging device 120, such as image resolution, lighting conditions, contrast, tint, etc. This provides standardized image conditions such that different patients in a database can be compared against each other to aid in ophthalmic diagnoses.

The ophthalmic cart 100 further includes a retinal imaging device 130. In one example, the retinal imaging device 130 includes an ultra-widefield retina imaging device, such as an Optos device for example. In one example, the ultra-widefield of the retinal imaging device 130 includes approximately 200 degrees of angular view, although the invention is not so limited. In one example, a fundus camera (such as external imaging device 120) may provide retina imaging, however such retina imaging may be more limited. For example, a fundus camera retina imaging may be limited to 45 degrees of angular view. It is desirable in selected data collection to have an ultra-widefield retina image to provide more inclusive retina data for a diagnosis.

Similar to the external imaging device 120 discussed above, different retinal imaging devices 130 may be used from one cart 100 to the next, provided that consistent ophthalmic data is obtained between devices 130. For example, if different retinal imaging devices 130 provide the same image resolution and/or color accuracy, etc., then different devices 130 are considered to provided standardized data. In one example, image capture settings are specified for any given retinal imaging device 130, such as image resolution, lighting conditions, angular field of view, contrast, tint, etc. This provides standardized image conditions such that different patients in a database can be compared against each other to aid in ophthalmic diagnoses.

The ophthalmic cart 100 further includes an optical coherence tomography (OCT) device 140. In one example the OCT 140 provides high-resolution 3D images that provide details of retinal tissue as well as below the surface of the retina. For example, a choroidal neovascular membrane in age-related macular degeneration can be detected from an OCT 140 as indicated by the presence of fluid between the neurosensory retina and the retinal pigment epithelium. The fluid at the interface is only imageable using OCT 140, or similar technology. In one example, the OCT is able to further image a morphology of a retinal detachment, which can indicate whether surgery is necessary as an emergency or within a few days. In one example OCT 140 provides detail on a capillary level beneath a surface of the retina and within the retina that may be useful in diagnosing a abnormal vasculature or vascular ischemia that may need to be treated. Other conditions apart from these examples may also be diagnosed using OCT 140 alone or in conjunction with other ophthalmic tools on the single cart 100.

Similar to the other ophthalmic tools discussed above, different OCT devices 140 may be used from one cart 100 to the next, provided that consistent ophthalmic data is obtained between OCT devices 140. For example, if one brand/configuration of OCT device 140 provides the same image resolution and/or color accuracy, etc. as other OCT devices 140 then different OCT devices 140 are considered to provided standardized data. In one example, image capture settings are specified for any given OCT device 140, such as image resolution, field of view, etc. In one example, a tissue density calibration factor is provided between different OCT devices 140 to provide consistent results between devices. This provides standardized image conditions such that different patients in a database can be compared against each other to aid in ophthalmic diagnoses.

FIG. 2 shows a Tono-Pen example of an intraocular pressure measurement device 110 as described above. The Tono-Pen 110 example of FIG. 2 includes an indentor 112, and an activation control 114. In operation, the indentor is placed in contact with a patient's eye, and the activation control 114 is pressed. In one example, a calibration value may be input by a user to standardize data across different tests from different patients and/or locations. Forces on the indentor are measured and provide correlated data to indicate intraocular pressure. In the example of FIG. 2, a transmission line 116 is used to power the device 110 and to transmit pressure data to the computer 106 of the cart 110. Other example devices 110 may be battery powered, and transmit data wirelessly. Additional devices for eye pressure monitoring may include wearable or implanted devices such as a contact lens with embedded sensors or a microsensor implanted inside the eye. Software on the computer 106 or from a remote server may provide instructions to an operator to correctly gather data with the Tono-Pen 110.

FIG. 3 shows a camera example of an external imaging device 120 as described above. A Volk Pictor device is shown, although the invention is not so limited. A viewing screen 124 is shown, along with controls 122 to manage image acquisition. A lens 126 is shown that is interfaced with the controls 122 to acquire images. A handle 128 makes this example of a fundus camera 120 particularly useful in the context of a mobile cart 100. As noted above, in one example, systems on the camera 120 are included to image fluorescence of the eye for evaluation of the cornea for epithelial defects. Particular ophthalmic data that the camera 120 provides, includes eyelid data, eyelash data, conjunctival data, and corneal data. Data is also collected with the camera 120 regarding the anterior chamber of the eye, the iris, lens, and pupil. Software on the computer 106 or from a remote server may provide instructions to an operator to correctly gather data with the camera 120.

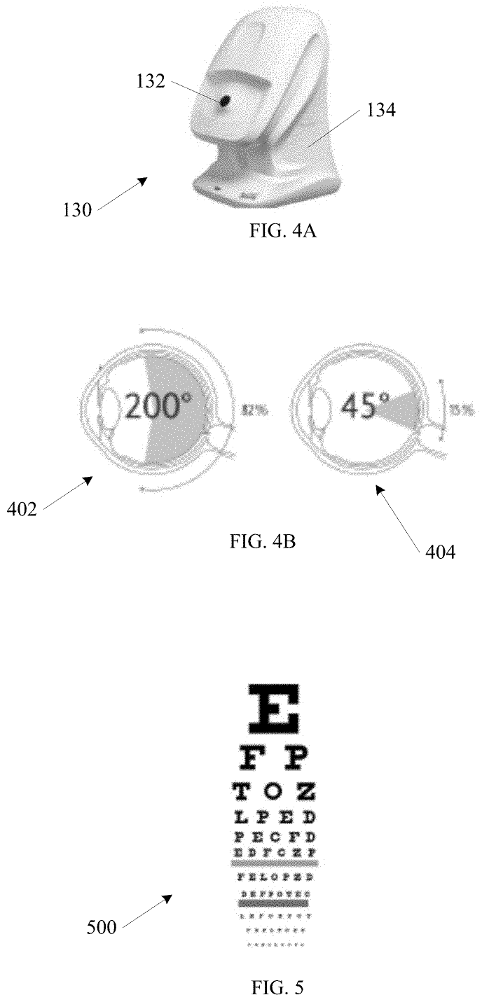

FIG. 4A shows an ultra-widefield retina imaging device example of a retina imaging device 130 as described above. An Optos device is shown, although the invention is not so limited. A data collection port 132 is shown, where a patient places their eye for imaging. A base 134 may contain imaging circuitry for operation of the retina imaging device 130. FIG. 4B illustrates an ultra-widefield angular view of the retina imaging device 130. Illustration 402 shows a 200 degree angular view that provides additional data for diagnosis over the more limited angular view shown in illustration 404. Particular ophthalmic data that the retina imaging device 130 provides, includes vitreous data, optic nerve data, macula data, retinal blood vessel data, and peripheral retina data.

In operation, a patient record is created that contains all ophthalmic data and images collected from the ophthalmic tools on the cart 100. The corresponding image analysis is tagged and indexed to the representative images, to include findings and diagnosis. The findings, diagnosis, and treatment recommendations are also tagged and indexed to the patients representative findings. By standardizing the data collection parameters, each new patient can be compared to all other patients in the database. Patient data and images are labeled in the system and findings associated with specific diagnoses and treatments. If other patient data and images are associated with a particular diagnosis, then a new patient whose data is similar to these patients can be more effectively and quickly diagnosed and triaged by an outside provider with minimal to no ophthalmic experience. In one example, artificial intelligence (AI) and machine learning (ML) algorithms may be used to quickly and consistently match other relevant patient records for diagnosis.

Data that AI/ML software may use for evaluation includes, but is not limited to blood vessel shape and color, macular shape, and other retina abnormalities, as well as anterior segment findings. AI/ML software then provides a differential diagnosis of possible conditions for the primary provider based on the constellation of data and findings. Data and imaging are immediately available via real-time alerts for review by a trained specialist to provide further guidance on diagnosis, treatment options, as well as determining the need for referral to a specialist on an urgent/emergent basis versus routine referral or no referral needed. AI/ML algorithms may also to be used for surgical guidance and assistance and will be discussed further below.

Although intraocular pressure, external imaging, and retinal imaging are discussed as collected ophthalmic data, other data may also be included to add to a database record. FIG. 5 shows an eye chart 500 that may be used to quantitatively measure character recognition and reading ability. Additional questions may be asked of a patient and recorded in the database. Questions may include, but are not limited to, diabetic condition, cardiovascular disease condition, autoimmune condition, glaucoma condition, macular degeneration condition, retinopathy, past eye surgeries, etc. Other questions may include, but are not limited to, time/date of problem, frequency of problem, any associated symptoms, pain, presence of floaters or flashes, decrease or loss of vision, whether the patient wears contact lenses, etc. Geographic location of a patient may also be included with a database record to facilitate geolocation and local physician referrals. Similar to other ophthalmic data, as discussed above, when possible, standardized data is preferred for any answer format. In this way, records can be sorted and compared to aid in future diagnoses, base treatment decisions, and referral patterns and locations.

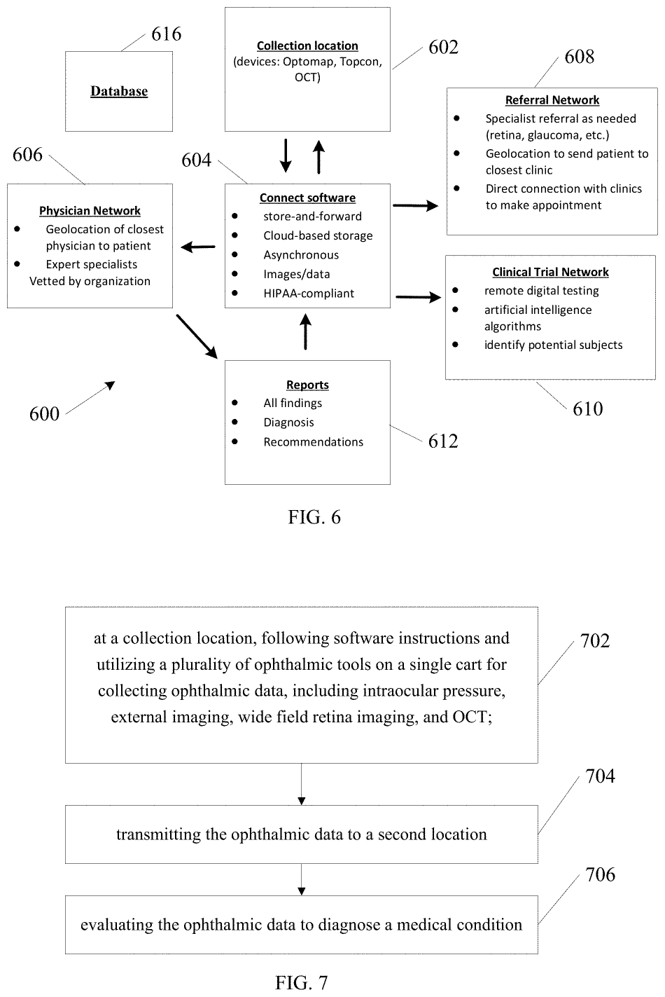

FIG. 6 shows a block diagram of software functionality for use with an ophthalmic cart according to one example. In block 602, a cart such as cart 100 is located. Examples of collection locations include, but are not limited to urgent care centers, emergency rooms, retail clinics (Walgreens, CVS, etc.), and primary care clinics. The cart 100 as described above, includes a plurality of ophthalmic tools and a computer with software for user instructions and data collection. In block 604, software may be included on a server at the same location, or a remote location to process data acquired in block 602. In one example, data is stored in a cloud-based configuration. Database 616 is shown in FIG. 6 and may be accessed from one or more of the blocks shown in the Figure. The processing of data in block 604 may be synchronous or asynchronous from the data collection in block 602. Asynchronous processing may allow for more efficient scheduling of time from different participants such as specialists, patients, and primary care physicians, etc.

In block 606 a network of physicians may be referred based on the data collected in block 602. In one example, physicians in block 606 are members of a network associated with block 604, and may be qualified or otherwise vetted. In block 608, other physicians or specialists may be referred from block 604. In one example, referrals in blocks 606 and 608 may be based on geographic data collected in block 602.

In block 610, the database 616 may be used to identify candidate for clinical trials of new drugs, procedures, devices, etc. Because patients typically see optometrists or primary care physicians with early stage disease (i.e. mild non-proliferative diabetic retinopathy) locally it is difficult for clinical trial projects to identify good candidates for the clinical trials, as most clinical trials are performed with specialists. Pharmaceutical advancements are investigating treatment at earlier time points in a disease process, thus, identifying and recruiting patients is important for these trials, as patients often are not referred into specialists until the disease has progressed to a later point and severity. Additionally, a first-level provider, such as an optometrist, may identify a potential candidate for a clinical trial with the platform allowing virtual consultation between the first-level provider, patient, and specialist to screen the patient prior to being enrolled in such a trial. The database 616 of data collected in block 602 provides a rich source of data to draw candidates from. Additionally, the standardized data as discussed above, provides accurate quantitative comparison to further sort from the database 616. The database 616 can enhance the accessibility and cost effectiveness of conducting clinical trials. In one example, AI/ML techniques may be used with the database 616 to sort records for potential in clinical trials.

Block 612 shows reports for individual patients, or a number of patients. In one example, a report is transmitted directly to a patient. In other examples, a report may be communicated back to the collection location from block 602 to be further communicated to the patient. In addition, the database will allow the patient to have access to their data, imaging and reports, thus allowing them to directly share their digital passport/medical record for second opinions or other consultations.

FIG. 7 shows an example method of use of ophthalmic data. In operation 702, software instructions are followed at a collection location. A plurality of ophthalmic tools are utilized on a single cart for collecting ophthalmic data. The data may include one or more of intraocular pressure, external imaging, ultra-widefield retina imaging, and optical coherence tomography (OCT). In operation 704, the ophthalmic data is transmitted to a second location. In operation 706, the ophthalmic data is evaluated to diagnose a medical condition and provide treatment recommendations or further referral to a specialist.

In one example, evaluation may be performed by a specialist or the use of AI/ML to triage and provide medical assessments and potential treatments and referral recommendations. For example, an anterior segment photo is taken, and patient reported symptoms of significant pain and decreased vision. AI/ML software identifies a cornea abrasion and software provides recommendations to a provider to treat with ophthalmic antibiotic ointment and patching of the eye. Follow-up appointment made with ophthalmic specialist within a week for follow-up.

As another example, a patient reports decreased vision in the emergency room. The patient may also report some symptoms of occasional fatigue and weakness. Ultra-wide field imaging shows swollen optic nerve in the left eye. AI/ML software identifies an optic nerve edema and its association with the other symptoms to recommend further testing with an Mill scan to evaluate for optic neuritis and possible brain lesions consistent with multiple sclerosis. Software identifies the diagnosis and presents best treatment options based on most recent medical literature. Appointment with neuro-ophthalmologist made for within 1 week.

In another example, a patient presents to primary care office with 20/20 vision in both eyes. Patient is a diabetic, so they undergo an ultra-wide field image of the retina. AI/ML identifies concern for proliferative diabetic retinopathy (PDR), an advanced diabetic complication that can lead to vision loss and blindness if not treated. Rather than just alerting that patient needs a referral to a retina specialist, the software alerts a retina surgeon virtually. The retina surgeon is able to connect virtually with the patient while the patient is in the primary care physician's office.

Statistically, only 50% of patients that are identified via telemedicine screening programs actually follow-up with a specialist. Using embodiments of devices and methods described in the present disclosure, a specialist is able to consult with the patient to discuss the disease process and severity to emphasize that it is imperative for the patient to follow-up in the office within the next few days for urgent treatment. With this combination of devices, software and workflows, we increase the compliance with follow-up after screening programs.

As noted above, example methods may involve evaluation of collected data at a later time and at a remote location. In other examples, evaluation of collected data may be in real time, or substantially real time, given data transmission speeds. One example of real time evaluation includes the use of devices and methods described for live surgery. For example, collecting live surgical 3D video allows an expert surgeon to view the surgery in real-time and interact via audio-video communication to provide expert guidance/assistance during surgery (tele-mentoring). The devices and methods described include software to allow remote pointers and real-time drawing to highlight parts of the surgery, including where to grab tissue, where to drain subretinal fluid, where to place a sclerotomy, and other surgical guidance tips and maneuvers to assist the surgeon.

In other examples, the collection of live surgical 3D video is transmitted from the primary operating room location to a remote second site using devices and methods described in the present disclosure. AI/ML software will analyze live surgical video to provide real-time surgical guidance and assistance. For example, software can identify the development of a retinal tear during surgery, highlight and mark the retinal tear, and offer recommendation to treat with laser. In another example, during surgery on a retinal detachment, the software, devices and methods described in the present disclosure will assess the subretinal fluid and determine whether to use a drainage retinotomy or perfluorocarbon fluid for fluid management, as well as the best place to create a drainage retinotomy for drainage of the subretinal fluid based on surgical video data combined with surgical outcomes data.

Additional example includes AI/ML to assess and highlight tissue edges such as epiretinal membrane (ERM) and internal limiting membrane (ILM). Devices such as OCT may be uniquely suited to determine tissue differences and to identify edges. An augmented reality overlay will highlight the tissue edges and adjust in real-time as the surgeon peels the tissue. Combined with surgical video data and surgical outcomes, the software, devices and methods described in the present disclosure can recommend need for additional tissue peeling to obtain the best outcome. This can also be used to assess a full-thickness macular hole, where the software will assess the size and configuration based on the live surgical data and the pre-operative optical coherence tomography (OCT) to provide recommendation on technique for surgeon to use to obtain the best outcomes (i.e. peel ILM to this surface area, or perform an ILM flap technique, or utilize C₃F₈ gas instead of SF₆ gas.).

### Example Computer System

Embodiments described herein may be implemented in one or a combination of hardware, firmware, and software. Embodiments may also be implemented as instructions stored on a machine-readable storage device, which may be read and executed by at least one processor to perform the operations described herein. A machine-readable storage device may include any non-transitory mechanism for storing information in a form readable by a machine (e.g., a computer). For example, a machine-readable storage device may include read-only memory (ROM), random-access memory (RAM), magnetic disk storage media, optical storage media, flash-memory devices, and other storage devices and media.

Examples, as described herein, may include, or may operate on, logic or a number of components, modules, or mechanisms. Modules may be hardware, software, or firmware communicatively coupled to one or more processors in order to carry out the operations described herein. Modules may hardware modules, and as such modules may be considered tangible entities capable of performing specified operations and may be configured or arranged in a certain manner. In an example, circuits may be arranged (e.g., internally or with respect to external entities such as other circuits) in a specified manner as a module. In an example, the whole or part of one or more computer systems (e.g., a standalone, client or server computer system) or one or more hardware processors may be configured by firmware or software (e.g., instructions, an application portion, or an application) as a module that operates to perform specified operations. In an example, the software may reside on a machine-readable medium. In an example, the software, when executed by the underlying hardware of the module, causes the hardware to perform the specified operations. Accordingly, the term hardware module is understood to encompass a tangible entity, be that an entity that is physically constructed, specifically configured (e.g., hardwired), or temporarily (e.g., transitorily) configured (e.g., programmed) to operate in a specified manner or to perform part or all of any operation described herein. Considering examples in which modules are temporarily configured, each of the modules need not be instantiated at any one moment in time. For example, where the modules comprise a general-purpose hardware processor configured using software; the general-purpose hardware processor may be configured as respective different modules at different times. Software may accordingly configure a hardware processor, for example, to constitute a particular module at one instance of time and to constitute a different module at a different instance of time. Modules may also be software or firmware modules, which operate to perform the methodologies described herein.

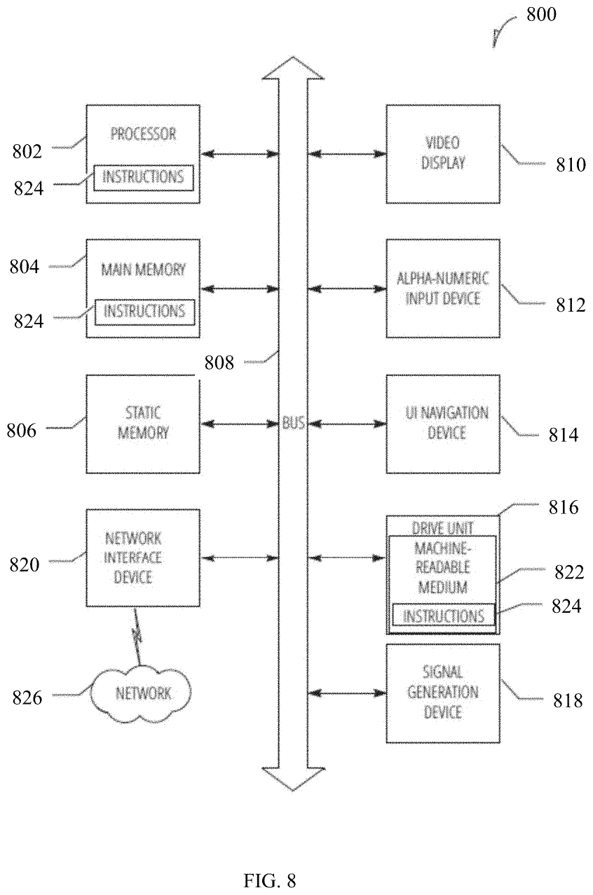

FIG. 8 is a block diagram illustrating a machine in the example form of a computer system 800, within which a set or sequence of instructions may be executed to cause the machine to perform any one of the methodologies discussed herein, according to an example embodiment. In alternative embodiments, the machine operates as a standalone device or may be connected (e.g., networked) to other machines. In a networked deployment, the machine may operate in the capacity of either a server or a client machine in server-client network environments, or it may act as a peer machine in peer-to-peer (or distributed) network environments. The machine may be an onboard vehicle system, wearable device, personal computer (PC), a tablet PC, a hybrid tablet, a personal digital assistant (PDA), a mobile telephone, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. Similarly, the term “processor-based system” shall be taken to include any set of one or more machines that are controlled by or operated by a processor (e.g., a computer) to individually or jointly execute instructions to perform any one or more of the methodologies discussed herein.

Example computer system 800 includes at least one processor 802 (e.g., a central processing unit (CPU), a graphics processing unit (GPU) or both, processor cores, compute nodes, etc.), a main memory 804 and a static memory 806, which communicate with each other via a link 808 (e.g., bus). The computer system 800 may further include a video display unit 810, an alphanumeric input device 812 (e.g., a keyboard), and a user interface (UI) navigation device 814 (e.g., a mouse). In one embodiment, the video display unit 810, input device 812 and UI navigation device 814 are incorporated into a touch screen display. The computer system 800 may additionally include a storage device 816 (e.g., a drive unit), a signal generation device 818 (e.g., a speaker), a network interface device 820, and one or more sensors (not shown), such as a global positioning system (GPS) sensor, compass, accelerometer, or other sensor.

The storage device 816 includes a machine-readable medium 822 on which is stored one or more sets of data structures and instructions 824 (e.g., software) embodying or utilized by any one or more of the methodologies or functions described herein. The instructions 824 may also reside, completely or at least partially, within the main memory 804, static memory 806, and/or within the processor 802 during execution thereof by the computer system 800, with the main memory 804, static memory 806, and the processor 802 also constituting machine-readable media.

While the machine-readable medium 822 is illustrated in an example embodiment to be a single medium, the term “machine-readable medium” may include a single medium or multiple media (e.g., a centralized or distributed database, and/or associated caches and servers) that store the one or more instructions 824. The term “machine-readable medium” shall also be taken to include any tangible medium that is capable of storing, encoding or carrying instructions for execution by the machine and that cause the machine to perform any one or more of the methodologies of the present disclosure or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including but not limited to, by way of example, semiconductor memory devices (e.g., electrically programmable read-only memory (EPROM), electrically erasable programmable read-only memory (EEPROM)) and flash memory devices; magnetic disks such as internal hard disks and removable disks; magneto-optical disks; and CD-ROM and DVD-ROM disks.

The instructions 824 may further be transmitted or received over a communications network 826 using a transmission medium via the network interface device 820 utilizing any one of a number of well-known transfer protocols (e.g., HTTP). Examples of communication networks include a local area network (LAN), a wide area network (WAN), the Internet, mobile telephone networks, plain old telephone (POTS) networks, and wireless data networks (e.g., Wi-Fi, 3G, and 4G LTE/LTE-A, 5G or WiMAX networks). The term “transmission medium” shall be taken to include any intangible medium that is capable of storing, encoding, or carrying instructions for execution by the machine, and includes digital or analog communications signals or other intangible medium to facilitate communication of such software.

To better illustrate the method and apparatuses disclosed herein, a non-limiting list of embodiments is provided here:

Example 1 includes an ophthalmic cart. The cart includes an intraocular pressure measurement device, an external imaging device, a wide field retina imaging device, and an optical coherence tomography imaging device. The cart also includes a display screen, at least one processor, and a storage device comprising instructions, which when executed by the at least one processor, configure the at least one processor to perform operations. The operations include recording ophthalmic images in a standardized format, comparing the ophthalmic images to a database of cataloged ophthalmic images in the standardized format and associated medical conditions, and presenting one or more suggested medical conditions from the comparison.

Example 2 includes the ophthalmic cart of Example 1, wherein the external imaging device is configured to use fluorescein dye to detect epithelial defects.

Example 3 includes the ophthalmic cart of any one of Examples, 1-2, wherein the wide field retina imaging device is configured to image a 200 degree field of view.

Example 4 includes the ophthalmic cart of any one of Examples, 1-3, wherein the intraocular pressure measurement device includes a tonopen device.

Example 5 includes a method. The method includes, at a collection location, following software instructions and utilizing a plurality of ophthalmic tools on a single cart for collecting ophthalmic data. The ophthalmic data includes intraocular pressure, external imaging, wide field retina imaging, and optical coherence tomography. The method also includes transmitting the ophthalmic data to a second location, and evaluating the ophthalmic data to diagnose a medical condition.

Example 6 includes the method of Example 5, further including storing at least a portion of the collected ophthalmic data in a database for diagnostic comparison.

Example 7 includes the method of any one of Examples, 5-6, wherein collecting ophthalmic data includes collecting standardized format wide field ophthalmic images, and comparing the standardized format wide field ophthalmic images with other wide field ophthalmic images in the standardized format and associated medical conditions in the database.

Example 8 includes the method of any one of Examples, 5-7, further including identifying ophthalmic data that corresponds to a clinical trial need, and listing potential candidates for the clinical trial based on the identified ophthalmic data.

Example 9 includes the method of any one of Examples, 5-8, wherein the identified ophthalmic data is recorded in a database in a standardized format.

Example 10 includes the method of any one of Examples, 5-9, wherein the identified standardized format ophthalmic data includes wide field retina imaging.

Example 11 includes the method of any one of Examples, 5-10, wherein evaluating the ophthalmic data is done in real time along with the collecting ophthalmic data.

Example 12 includes the method of any one of Examples, 5-11, wherein evaluating the ophthalmic data is performed by an artificial intelligence of machine learning algorithm.

Example 13 is a non-transitory computer readable medium comprising instructions, which when executed by at least one processor, configure the at least one processor to perform operations. The operations include recording ophthalmic data to a database, including intraocular pressure data, external imaging data and ophthalmic images in a standardized format. The operations further include comparing the standardized format ophthalmic images to other ophthalmic images in the standardized format in the database and associated medical conditions, and presenting one or more suggested medical conditions based on the comparison, the intraocular pressure data, and the external imaging data.

Example 14 includes the non-transitory computer readable medium of example 13, further including instructions to perform operations comprising identifying records from the database that corresponds to a clinical trial need, and listing potential candidates for the clinical trial based on the identified records.

Example 15 includes the non-transitory computer readable medium of any one of examples 13-14, wherein the ophthalmic images in the standardized format includes wide field retina images of approximately 200 degrees in a standardized format.

Example 16 includes the non-transitory computer readable medium of any one of examples 13-15, further including instructions to perform operations comprising transmitting the ophthalmic data to a second location different than a data collection location for diagnosis.

Example 17 includes the non-transitory computer readable medium of any one of examples 13-16, further including instructions to perform operations comprising utilizing a plurality of ophthalmic tools on a single cart at the collection location to collect the ophthalmic data.

Example 18 is a system. The system includes at least one processor and a storage device comprising instructions, which when executed by the at least one processor, configure the at least one processor to perform operations. The operations include recording ophthalmic data to a database, including intraocular pressure data, external imaging data, and ophthalmic images in a standardized format. The operations further include comparing the standardized format ophthalmic images to other ophthalmic images in the standardized format in the database and associated medical conditions, and presenting one or more suggested medical conditions based on the comparison, the intraocular pressure data, and the external imaging data.

Example 19 includes the system of example 18, further including instructions to perform operations comprising identifying records from the database that corresponds to a clinical trial need, and listing potential candidates for the clinical trial based on the identified records.

Example 20 includes the system of any one of examples 18-19, wherein the ophthalmic images in a standardized format includes wide field retina images of approximately 200 degrees in a standardized format.

Example 21 includes the system of any one of examples 18-20, further including instructions to perform operations comprising transmitting the ophthalmic data to a second location different than a data collection location for diagnosis.

Example 22 includes the system of any one of examples 18-21, further including instructions to perform operations comprising utilizing a plurality of ophthalmic tools on a single cart at the collection location to collect the ophthalmic data.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Although an overview of the inventive subject matter has been described with reference to specific example embodiments, various modifications and changes may be made to these embodiments without departing from the broader scope of embodiments of the present disclosure. Such embodiments of the inventive subject matter may be referred to herein, individually or collectively, by the term “invention” merely for convenience and without intending to voluntarily limit the scope of this application to any single disclosure or inventive concept if more than one is, in fact, disclosed.

The embodiments illustrated herein are described in sufficient detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

As used herein, the term “or” may be construed in either an inclusive or exclusive sense. Moreover, plural instances may be provided for resources, operations, or structures described herein as a single instance. Additionally, boundaries between various resources, operations, modules, engines, and data stores are somewhat arbitrary, and particular operations are illustrated in a context of specific illustrative configurations. Other allocations of functionality are envisioned and may fall within a scope of various embodiments of the present disclosure. In general, structures and functionality presented as separate resources in the example configurations may be implemented as a combined structure or resource. Similarly, structures and functionality presented as a single resource may be implemented as separate resources. These and other variations, modifications, additions, and improvements fall within a scope of embodiments of the present disclosure as represented by the appended claims. The specification and drawings are, accordingly, to be regarded in an illustrative rather than a restrictive sense.

The foregoing description, for the purpose of explanation, has been described with reference to specific example embodiments. However, the illustrative discussions above are not intended to be exhaustive or to limit the possible example embodiments to the precise forms disclosed. Many modifications and variations are possible in view of the above teachings. The example embodiments were chosen and described in order to best explain the principles involved and their practical applications, to thereby enable others skilled in the art to best utilize the various example embodiments with various modifications as are suited to the particular use contemplated.

It will also be understood that, although the terms “first,” “second,” and so forth may be used herein to describe various elements, these elements should not be limited by these terms. These terms are only used to distinguish one element from another. For example, a first contact could be termed a second contact, and, similarly, a second contact could be termed a first contact, without departing from the scope of the present example embodiments. The first contact and the second contact are both contacts, but they are not the same contact.

The terminology used in the description of the example embodiments herein is for the purpose of describing particular example embodiments only and is not intended to be limiting. As used in the description of the example embodiments and the appended examples, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

1. An ophthalmic cart, comprising:
an intraocular pressure measurement device;
an external imaging device;
a wide field retina imaging device;
an optical coherence tomography imaging device;
a display screen;
at least one processor;
a storage device comprising instructions, which when executed by the at least one processor, configure the at least one processor to perform operations comprising:
recording ophthalmic images in a standardized format;
comparing the ophthalmic images to a database of cataloged ophthalmic images in the standardized format and associated medical conditions; and
presenting one or more suggested medical conditions from the comparison.

2. The ophthalmic cart of claim 1, wherein the external imaging device is configured to use fluorescein dye to detect epithelial defects.

3. The ophthalmic cart of claim 1, wherein the wide field retina imaging device is configured to image a 200 degree field of view.

4. The ophthalmic cart of claim 1, wherein the intraocular pressure measurement device includes a tonopen device.

5. A method, comprising:
at a collection location, following software instructions and utilizing a plurality of ophthalmic tools on a single cart for collecting ophthalmic data, including:
intraocular pressure;
external imaging;
wide field retina imaging;
optical coherence tomography;
transmitting the ophthalmic data to a second location; and
evaluating the ophthalmic data to diagnose a medical condition.

6. The method of claim 5, further including storing at least a portion of the collected ophthalmic data in a database for diagnostic comparison.

7. The method of claim 6, wherein collecting ophthalmic data includes collecting standardized format wide field ophthalmic images, and comparing the standardized format wide field ophthalmic images with other wide field ophthalmic images in the standardized format and associated medical conditions in the database.

8. The method of claim 5, further including identifying ophthalmic data that corresponds to a clinical trial need, and listing potential candidates for the clinical trial based on the identified ophthalmic data.

9. The method of claim 8, wherein the identified ophthalmic data is recorded in a database in a standardized format.

10. The method of claim 9, wherein the identified standardized format ophthalmic data includes wide field retina imaging.

11. The method of claim 8, wherein evaluating the ophthalmic data is done in real time along with the collecting ophthalmic data.

12. The method of claim 8, wherein evaluating the ophthalmic data is performed by an artificial intelligence of machine learning algorithm.

13. A non-transitory computer readable medium comprising instructions, which when executed by at least one processor, configure the at least one processor to perform operations comprising:
recording ophthalmic data to a database, including:
intraocular pressure data;
external imaging data;
ophthalmic images in a standardized format;
comparing the standardized format ophthalmic images to other ophthalmic images in the standardized format in the database and associated medical conditions; and
presenting one or more suggested medical conditions based on the comparison, the intraocular pressure data, and the external imaging data.

14. The non-transitory computer readable medium of claim 13, further including instructions to perform operations comprising identifying records from the database that corresponds to a clinical trial need, and listing potential candidates for the clinical trial based on the identified records.

15. The non-transitory computer readable medium of claim 13, wherein the ophthalmic images in the standardized format includes wide field retina images of approximately 200 degrees in a standardized format.

16. The non-transitory computer readable medium of claim 13, further including instructions to perform operations comprising transmitting the ophthalmic data to a second location different than a data collection location for diagnosis.

17. The non-transitory computer readable medium of claim 16, further including instructions to perform operations comprising utilizing a plurality of ophthalmic tools on a single cart at the collection location to collect the ophthalmic data.

18. A system comprising:
at least one processor;
a storage device comprising instructions, which when executed by the at least one processor, configure the at least one processor to perform operations comprising:
recording ophthalmic data to a database, including:
intraocular pressure data;
external imaging data;
ophthalmic images in a standardized format;
comparing the standardized format ophthalmic images to other ophthalmic images in the standardized format in the database and associated medical conditions; and
presenting one or more suggested medical conditions based on the comparison, the intraocular pressure data, and the external imaging data.

19. The system of claim 18, further including instructions to perform operations comprising identifying records from the database that corresponds to a clinical trial need, and listing potential candidates for the clinical trial based on the identified records.

20. The system of claim 18, wherein the ophthalmic images in a standardized format includes wide field retina images of approximately 200 degrees in a standardized format.

21. The system of claim 18, further including instructions to perform operations comprising transmitting the ophthalmic data to a second location different than a data collection location for diagnosis.

22. The system of claim 21, further including instructions to perform operations comprising utilizing a plurality of ophthalmic tools on a single cart at the collection location to collect the ophthalmic data.
